(19) United States
(12) Patent Application Publication
(10) Pub. No.: US 2024/0139093 A1
(43) Pub. Date: May 2, 2024

(54) EXTEMPORANEOUS PREPARATION AND COSMETIC METHOD FOR STRAIGHTENING HAIR USING SAID PREPARATION

(71) Applicant: NOVANTIC COSMETICS, STRASBOURG (FR)

(72) Inventor: FATIMA ERRAKHAOUI-MURA, STRASBOURG (FR)

(21) Appl. No.: 18/548,234

(22) PCT Filed: Mar. 4, 2022
(86) PCT No.: PCT/FR2022/050389

§ 371 Date: Aug. 29, 2023

(30) Foreign Application Priority Data
Mar. 4, 2021 (FR) FR2102096

(51) Int. Cl. A61K 8/9783; A61K 8/02; A61K 8/04; A61K 8/19; A61K 8/92; A61Q 5/04

(52) U.S. Cl. CPC A61K 8/9783; A61K 8/0216; A61K 8/04; A61K 8/19; A61K 8/922; A61Q 5/04; A61K 2800/591

(57) ABSTRACT

The present invention relates to an extemporaneous preparation, comprising, in a physiologically acceptable aqueous medium, an extract of tamarisk galls in the form of ground gall grains and copper oxide powder. The invention also relates to a cosmetic method for straightening hair using this preparation.

### SUBJECT OF THE INVENTION

The present invention relates to an extemporaneous preparation comprising, in a physiologically acceptable aqueous support, Tamaris gall extract in the form of ground gall grains and copper oxide powder. The invention also relates to a cosmetic process for straightening the hair using this preparation.

### BACKGROUND OF THE INVENTION

Various techniques have been proposed for straightening or relaxing curly or frizzy hair. In general, the hair can be straightened temporarily and reversibly by mechanical shaping, notably using a straightening iron. For a longer-lasting result, designed to withstand the action of washing with water or shampoo, it is necessary to modify the physicochemical properties of keratin by means of a reducing agent which can break its disulfide bridges (cystine), generally after application of a basifying agent designed to swell the hair and open the cuticle. Once the disulfide bridges have been broken, the keratin chains are able to slide over each other. Once the hair has been shaped, these bridges are then reconstituted via the action of an oxidizing agent, generally in the presence of an acid that reduces the swelling of the hair and smooths the cuticle. These three successive steps of reduction, smoothing and fixing typically use thioglycolic acid or salts or esters thereof, or sulfites or bisulfites, as the reducing agent, ammonia as the basifying agent and hydrogen peroxide as the oxidizing agent. However, these reagents generate a very strong odor which may be problematic when used in a hairdressing salon, not only for the customer on whom these products are applied, but also for other customers. On very curly or even frizzy hair, alkaline agents such as sodium, potassium or guanidine hydroxide are preferred, providing a longer-lasting effect. The oxidizing agent is then replaced with a neutralizing shampoo, as the oxidation step is not necessary. This solution does not generate an unpleasant odor, but has the drawback of severely irritating the scalp and impairing the hair, which becomes rough to the touch and brittle to the point of crumbling.

Another problem with the techniques known to date is that their repeated application to the hair leads, over time, to a significant change in the hair's behavior, in particular with regard to its ability to be properly dyed afterwards.

In addition to these professional techniques, there are traditional hair straightening methods used mainly in North Africa, such as Tunisian merdouma, which is a preparation based on gall nuts, iron filings, cloves, plant oil and optionally henna, applied to the hair for simultaneous straightening and dyeing.

Gall nuts are rigid-walled excrescences resulting from the proliferation of plant tissue in response to irritation caused by the sting of certain parasites on plants such as oak, chestnut or shrubs of the Tamaris or Tamarix genus. These excrescences may form on any plant organ, such as the leaves, flowers, buds, fruit or stems. Tamaris gall is produced by an insect (cynips) and is known for its richness in tannins and its dyeing properties, which justifies its use in the abovementioned recipe, but also in similar compositions, in which it is combined with other plants (MA21838) or in which copper oxide is used instead of iron filings (FR 2672491 and US 2003/185785).

Nevertheless, there is still a need to develop a preparation for permanent hair straightening that is effective on wavy, curly or frizzy hair, and non-aggressive to the hair, in the sense that it leaves the hair soft, supple and shiny.

After extensive research, the inventors have developed a preparation that can meet this need.

### SUMMARY OF THE INVENTION

The subject of the invention is an extemporaneous preparation comprising, in a physiologically acceptable aqueous support, Tamaris gall extract in the form of ground gall grains and copper oxide powder.

The subject of the invention is also a cosmetic hair straightening process, comprising the following steps:
(a) applying the abovementioned preparation to the hair,
(b) rinsing the hair to remove the preparation, optionally followed by drying the hair, steps (a) and (b) possibly being repeated,
(c) applying to the hair an oily and/or water-based conditioning composition,
(d) washing the hair.

### DETAILED DESCRIPTION

The present invention relates to an extemporaneous preparation, i.e. a preparation obtained by mixing at least two separate compositions, immediately prior to use. This preparation is particularly suitable for application to the hair and the scalp for the purpose of conditioning and, even more precisely, for straightening the hair.

This preparation contains two key ingredients, namely Tamaris gall extract in the form of ground gall grains and copper oxide powder.

The galls may be derived from a Tamaris species notably chosen from: Tamaris gallica, Tamaris africana, Tamaris orientalis, Tamaris articulata and Tamaris aphylla, preferably Tamaris orientalis. To obtain the gall extract according to the invention, galls are generally immersed in an oil bath, at a temperature preferably between 180 and 240° C., for a period ranging from 30 minutes to one hour, then removed from the bath so as to grind them to the desired size, and the powder thus obtained is optionally sieved, and is preferably stored under vacuum. The term “oil” means a compound which is liquid at room temperature (25° C.) and atmospheric pressure (10⁵ Pa) and which, when introduced at a rate of at least 1% by weight into water at 25° C., is not at all soluble in water, or is soluble to an extent of less than 10% by weight, relative to the weight of oil introduced into the water. As oils that may be used for gall extraction, mention may be made of plant oils, branched or unsaturated fatty alcohols, fatty esters, hydrocarbons of plant or mineral origin, silicone oils and mixtures thereof, preferably plant oils. Mention may notably be made of wheat germ oil, sunflower oil, argan oil, broccoli oil, hibiscus oil, coriander oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, lavender oil, borage oil, millet oil, barley oil, quinoa oil, rye oil, safflower oil, candlenut oil, passionflower oil, rose hip oil, Echium oil, camelina oil, camellia oil or mixtures thereof. Tamaris gall extract thus contains at least one of the abovementioned plant oils, advantageously chosen from olive oil, argan oil, jojoba oil or castor oil or mixtures thereof, which coats and/or impregnates the ground gall grains. Even more preferentially, the plant oil is a mixture of olive oil and argan oil.

This gall extract is mixed extemporaneously with a copper oxide powder, which has been shown to improve hair straightening and to facilitate shaping. According to the present invention, the term “copper oxide powder” means a powder predominantly containing copper in the form of metal oxide, i.e. copper(I) oxide and/or copper(II) oxide. In particular, the copper powder used according to the invention may predominantly or even exclusively contain copper(II) oxide. It thus has a more or less dark red color. In a preferred embodiment of the invention, the copper oxide powder contains at least 90% by weight, preferably at least 95% by weight, or even at least 97% by weight, of copper oxide, as determined by wavelength-dispersive X-ray fluorescence spectrometry (WDX-FSX) and X-ray diffraction, and not more than 5% by weight of at least one other compound chosen from metals, salts thereof and alloys thereof, and/or silicon-based compounds. The copper oxide powder may represent from 5% to 25% by weight, preferably from 8% to 20% by weight and more preferentially from 10% to 15% by weight, relative to the weight of the Tamaris gall extract, so as to achieve the desired hair straightening effect without weakening the hair.

The preparation according to the invention is obtained by mixing the gall extract and copper oxide powder described previously in a physiologically acceptable aqueous medium. The term “physiologically acceptable” means that this medium is suitable for application to the hair and the scalp, and is nontoxic thereto. The mixing of the gall extract, copper oxide powder and the aqueous medium may take place in any order, but it is preferred that the gall extract is first intimately mixed with the copper oxide powder before being added to the aqueous medium. This aqueous medium is usually brought to a temperature from 80 to 100° C., preferentially to the boiling point, before or after, preferably before, the addition of the abovementioned mixture, and kept stirring until a creamy or pasty semi-solid composition of homogeneous consistency is obtained, for example using a blender-type mixer.

The aqueous medium mentioned previously necessarily comprises water. In one embodiment, it may be a mixture of water and of at least one water-soluble solvent chosen from C₁-C₄ alcohols, such as ethanol, isopropanol, tert-butanol or n-butanol; polyols such as glycerol, propylene glycol and polyethylene glycols; and mixtures thereof. As a variant or in addition, the aqueous medium may comprise at least one oil chosen, for example, from those mentioned previously. This aqueous medium based on water and optionally alcohol, polyol and/or oil may also contain various cosmetic ingredients listed in the CTFA Dictionary, such as surfactants, thickening and/or gelling agents, pigments, fillers, pH adjusters, preserving agents and mixtures thereof.

In a preferred embodiment of the invention, the extemporaneous preparation contains at least 90%, or even at least 95% and, better still, 100% of a mixture of water, Tamaris gall extract and copper oxide powder, relative to the total weight of this preparation.

The present invention also relates to a cosmetic hair straightening process using the abovementioned preparation and comprising precisely the following steps:
(a) applying the preparation described previously to the hair,
(b) rinsing the hair to remove the preparation, optionally followed by drying the hair, steps (a) and (b) possibly being repeated,
(c) applying to the hair an oily composition and/or a water-based conditioning composition,
(d) washing the hair.

In one embodiment of the invention, said process comprises the preliminary steps consisting in manufacturing the preparation used in step (a), comprising:
(a1) mixing ground Tamaris galls, optionally coated and/or impregnated with at least one oil, with copper oxide powder,
(a2) adding this mixture to water previously brought to a temperature of 80 to 100° C., to obtain a dispersion, and
(a3) mixing this dispersion, advantageously in a mixer, to obtain a semi-solid composition.
   This process may also comprise the addition of one or more cosmetic ingredients as defined previously in at least one of the steps (a1) to (a3).

In order to facilitate the implementation of the straightening process according to the invention, the various compositions that are useful for its execution may be combined in a multi-packaging device or “kit” comprising:
a first package containing Tamaris gall extract and, optionally, at least one cosmetic ingredient,
a second package containing copper oxide powder and optionally at least one cosmetic ingredient,
a third package containing an oily composition and/or a fourth package containing a water-based conditioning composition,
optionally, a fifth package containing a shampoo.

Each of the first and second packages may be independently in the form of a jar, can, sachet or salt shaker-type dispenser, for instance. Moreover, each of the third, fourth and fifth packages may be in the form of a tube, bottle or a pump bottle, notably. The first and second packages may contain an amount of product which, after mixing, allows the preparation according to the invention to be obtained in an amount necessary and sufficient for a single application, two, three or four applications, or even a greater number of applications. The other packages may also contain an amount of composition that is suitable or unsuitable for a single implementation of the process according to the invention.

The abovementioned kit may also contain one or more devices allowing the application of one or more of the compositions contained in the kit, notably a brush and/or gloves. As a variant or in addition, the kit may contain a piece of fabric or nonwoven suitable for wrapping the head.

In step (a) of the process according to the invention, the extemporaneous preparation is generally applied to previously washed and rinsed hair. It may be applied to wet or dry hair, and preferably to dry hair. The hair to be treated may be more or less wavy, curly or frizzy. The effective amount used depends on the hair's length and condition. It is generally applied to the hair with a brush and left on for a period ranging from 10 minutes to 16 hours. The leave-on time may be longer or shorter, depending on the length of the hair and on whether the hair is placed under a heating hood or dried naturally. As it dries, the extemporaneous preparation hardens and generally forms a shell on the head, while at the same time giving off a pleasant warmth. If necessary, a microfiber headband or silk scarf may be applied to the hair before drying, so as to perfect the result.

The hair is then rinsed in step (b) with clear water or a mild shampoo, or a combination thereof. These two steps are generally repeated up to six times, preferably one to three times, when the process according to the invention is performed on the hair for the first time. They do not generally need to be repeated for subsequent applications of the process intended to treat new growth.

In order to relax the hair and allow it to be shaped, the process according to the invention then comprises a step (c) consisting in applying an oily composition and/or a water-based conditioning composition to the hair.

The oily composition comprises at least one oil chosen from those listed previously. This is preferably a plant oil and more preferentially an oil chosen from olive oil, argan oil, jojoba oil, castor oil and mixtures thereof. This composition may also contain one or more additional fatty substances, such as fatty-phase gelling agents, waxes, pasty fatty substances or mixtures thereof, and also optionally one or more liposoluble or lipodispersible additives. It may also be in the form of a water-in-oil emulsion containing an aqueous phase consisting of water or comprising water and at least one alcohol and/or polyol. As a variant or in addition, the aqueous phase may comprise at least one water-soluble or water-dispersible cosmetic ingredient. In the case where it is in the form of an emulsion, this oily composition may also contain one or more emulsifiers.

This oily composition may be replaced with, or followed by, the application of a water-based hair conditioning composition. For the purposes of this description, the term “conditioning” means improving at least one hair property chosen from: combability, disentangling ability, softness, suppleness, sheen and manageability. This composition may be in the form of an aqueous solution or dispersion, or in the form of an oil-in-water (O/W) emulsion, having a liquid or semi-liquid consistency, a soft creamy consistency or a solid consistency.

The conditioning composition generally comprises at least one conditioning agent chosen from plant extracts, cationic polymers, cationic surfactants and mixtures thereof, notably quaternary ammonium salts such as behentrimonium chloride and amino silicones denoted by the INCI names amodimethicone, bis-aminopropyl dimethicone, trimethylsilyl amodimethicone and PEG-7 aminodimethicone. This conditioning composition may also comprise at least one customary cosmetic ingredient, notably chosen from nonionic surfactants, cationic surfactants, anionic surfactants, amphoteric surfactants; sunscreens; vitamins; antidandruff agents, antiseborrheic agents, hair loss inhibitors and/or hair regrowth agents; antioxidants; nacreous agents and/or opacifiers; pigments; fillers; sequestrants; thickeners; fragrances; preserving agents; and mixtures thereof.

The conditioning composition used according to the invention is in particular a composition based on neutral henna (Cassia auriculata or obovata or italica) or a dye composition based on natural henna (Lawsonia inermis), obtained by dissolving henna powder in water and optionally adding one or more cosmetic ingredients notably chosen from other plant extracts, notably plant protein hydrolyzates, oils, antioxidants, fragrances and mixtures thereof, to obtain a creamy or pasty semi-solid composition. Natural henna allows the hair color obtained via the process to be modulated, insofar as the gall extract used in step (a) has the effect of giving the hair a very dark hue. This henna mask, whether neutral or natural, also has the advantage of coating the hair fiber and improving its suppleness. The leave-on time for this mask may be between 4 h and 16 h, and preferably between 8 h and 14 h, before being washed out with water.

The hair is finally washed, generally using a mild shampoo, so as to remove residues of the compositions applied previously.

The process described above allows wavy, curly or even frizzy hair to be straightened very effectively, without damaging it. The result is a permanent effect, in the sense that only new growth needs to be treated.

### BRIEF DESCRIPTION OF THE FIGURES

FIG. 1 is a photograph showing the appearance of a lock of tight, wavy hair before and after treatment.

FIG. 2 is a photograph showing the appearance of a lock of hair with tight, abundant curls before and after treatment.

FIG. 3 is a photograph showing the appearance of a subject's hair before treatment according to the invention.

FIG. 4 is a photograph showing the appearance of the same subject's hair after treatment according to the invention.

FIG. 5 represents the scale used to characterize the degree of frizziness of locks of hair.

### EXAMPLES

The invention will be better understood in the light of the following examples, which are given purely by way of illustration and are not intended to limit the scope of the invention, defined by the appended claims.

### Example 1: Extemporaneous Preparation

Tamaris orientalis galls were cooked in a bath containing a mixture of olive oil and argan oil. The galls were then removed from the bath, dried, cooled, finely ground and the powder obtained was sieved and placed under vacuum to form the gall extract used according to the invention.

To make the extemporaneous preparation according to the invention, 600 g of gall extract were homogeneously mixed with 60 g of copper oxide powder, and this mixture was then added to 600 g of boiling water with stirring. The whole mixture was passed through a blender to obtain a homogeneous-looking paste.

### Example 2: Ex-Vivo Sensory Evaluation

The efficacy of the composition according to Example 1 on straightening the hair was evaluated, as was the appearance of the hair on conclusion of the straightening process.

To do this, 20 locks of hair with varying degrees of frizziness were prepared, first washed with a neutral shampoo washing (Mixa® Bébé, 1 ml/2 g hair) for 10 seconds, then rinsed with water for 30 seconds and dried naturally.

The paste of Example 1 was then applied to the locks as a mask, for 8 hours. The locks were covered with a hairband to accelerate drying. They were then rinsed thoroughly with water and dried with a hair dryer without blow-drying. This step was repeated twice.

The locks were then massaged with argan oil, before applying thereto a henna mask obtained by mixing henna powder with warm water. After a leave-on time of 8 hours, the locks were rinsed thoroughly with water. They were then washed again using the same neutral shampoo and dried naturally.

The locks were evaluated before application of the straightening preparation according to Example 1 and on conclusion of the treatment. Specifically, the following were evaluated:
the maximum length of the locks, which was measured after manual extension (before performing the straightening process) or without manual extension (on conclusion of the process),
the state of frizziness of locks, with a score from 1 (straight hair) to 10 (very, very tight curls), based on the frizziness scale shown in FIG. 5,
the general appearance of the locks, which were photographed, and
the softness of the locks to the touch, scoring them from 1 (rough) to 10 (soft).

The average of the results obtained on the 20 locks was calculated.

The normality of the data was verified using the Shapiro-Wilk test, and Student's t test was used to compare the averages obtained before and after treatment.

The straightening efficacy (in %) was determined by calculating the value (Lt−Lo)/Lo×100, where Lo is the length of the lock before treatment and Lt is the length of the lock after treatment.

The results are given in Table 1 below.

TABLE 1

| | | Straightening | |
|---|---|---|---|
| Average | Average | efficacy | |
| Lo | Lt | (%) | p |
| 20.4 ± 0.9 | 19.9 ± 0.8 | 97.6 ± 0.3 | <0.0001 |

The treatment according to the invention allowed the hair to be straightened considerably, with the hair, when at rest, reaching practically the length it had before treatment and stretched to the maximum.

The straightening efficacy was evaluated by calculating the value So−St, where So is the state of frizziness before treatment and St the state of frizziness after treatment.

The results are given in Table 2 below.

TABLE 2

| Average | Average | Straightening | |
|---|---|---|---|
| So | St | efficacy | p |
| 5.6 ± 0.3 | 1.8 ± 0.2 | 3.8 ± 0.2 | <0.0001 |

The treatment according to the invention allowed the hair to move from the “tight, dense curl” medium state of frizziness to the “wavy” state.

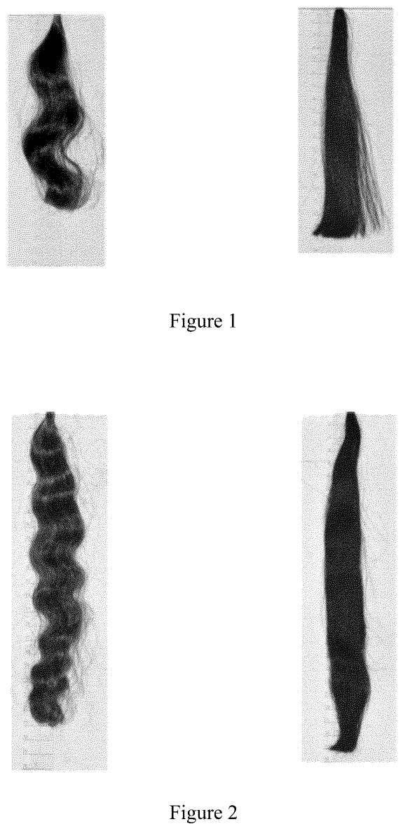

The attached Figures illustrate the result obtained on two locks of hair, with a pre-treatment state of frizziness of 4 (FIGS. 1) and 7 (FIG. 2), respectively.

Finally, the variation in hair softness was evaluated by calculating the value Dt−Do, where Do is the pre-treatment softness score and Dt the post-treatment softness score.

The results are given in Table 3 below.

TABLE 3

| Average | Average | Softness | |
|---|---|---|---|
| Do | Dt | variation | p |
| 5.1 ± 0.3 | 5.4 ± 0.4 | 0.3 ± 0.3 | 0.3569 |
| | | | (not significant) |

The treatment according to the invention did not significantly modify the softness of the hair; it is thus not aggressive to the hair.

### Example 3: In-Vivo Sensory Evaluation

The efficacy of the preparation according to the invention on straightening a subject's hair was evaluated, as was the appearance of the hair on conclusion of the straightening process.

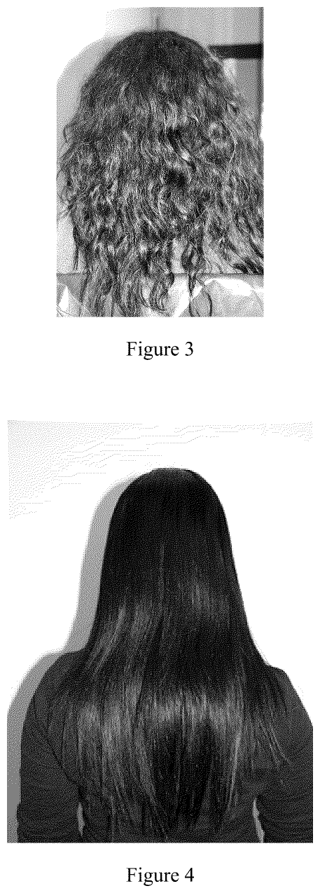

Before treatment, the subject's hair was made up of well-formed loose curls, corresponding to the state of frizziness No. 5. The appearance of the hair after washing and blow-drying is shown in FIG. 3.

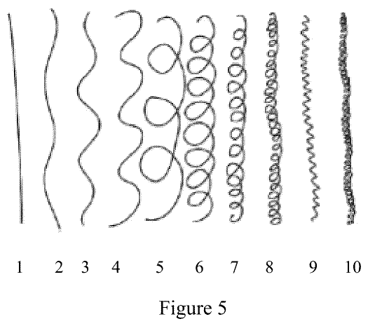

The preparation according to Example 1 was applied three times in succession (over three consecutive days), each application being followed by rinsing with water. A conditioning mask was then applied to the hair, which was washed using a mild shampoo. After about ten days, this period allowing the hair to evacuate the excess product, the hair was perfectly straight and shiny, as shown in FIG. 4.

1-11. (canceled).

12. An extemporaneous preparation comprising, in a physiologically acceptable aqueous support, a Tamaris gall extract in the form of ground gall grains and copper oxide powder.

13. The preparation as claimed in claim 12, characterized in that the galls are obtained from a Tamaris species chosen from: Tamaris gallica, Tamaris africana, Tamaris orientalis, Tamaris articulata and Tamaris aphylla, preferably Tamaris orientalis.

14. The preparation as claimed in claim 12, characterized in that the Tamaris gall extract is obtained by cooking in an oil bath and grinding the Tamaris galls.

15. The preparation as claimed in claim 12, characterized in that the Tamaris gall grain extract is coated and/or impregnated with at least one plant oil, selected from wheat germ oil, sunflower oil, argan oil, broccoli oil, hibiscus oil, coriander oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, lavender oil, borage oil, millet oil, barley oil, quinoa oil, rye oil, safflower oil, candlenut oil, passionflower oil, rose hip oil, Echium oil, camelina oil, camellia oil or mixtures thereof, preferably from olive oil, argan oil, jojoba oil, castor oil or mixtures thereof.

16. The preparation as claimed in claim 15, characterized in that the plant oil is formed from a mixture of olive oil and argan oil.

17. The preparation as claimed in claim 12, characterized in that the copper oxide powder contains at least 95% copper in the form of copper(I) oxide and/or copper(II) oxide, as determined by wavelength-dispersive X-ray fluorescence spectrometry, and not more than 5% by weight of at least one other compound selected from metals, salts thereof and alloys thereof, and/or silicon-based compounds.

18. The preparation as claimed in claim 12, characterized in that the copper oxide powder predominantly or exclusively contains copper(II) oxide.

19. The preparation as claimed in claim 12, characterized in that the copper oxide powder represents from 5% to 25% by weight, relative to the weight of the Tamaris gall extract.

20. A cosmetic process for straightening the hair, comprising the following steps:
(a) applying the preparation as claimed in claim 12 to the hair,
(b) rinsing the hair to remove the preparation, optionally followed by drying the hair, steps (a) and (b) optionally being repeated,
(c) applying to the hair an oily composition and/or a water-based conditioning composition,
(d) washing the hair.

21. The process as claimed in claim 20, characterized in that step (a) comprises the following substeps:
(a1) mixing ground Tamaris galls, optionally coated and/or impregnated with at least one oil, with copper oxide powder,
(a2) adding this mixture to water previously brought to a temperature of 80 to 100° C., to obtain a dispersion, and
(a3) mixing this dispersion, advantageously in a mixer, to obtain a semi-solid composition.

22. The process as claimed in claim 21, characterized in that it also comprises the addition of one or more cosmetic ingredients in at least one of the steps (a1) to (a3).
